SEQUENCE LISTING (8 sequences)

SEQ ID NO: 1 - DNA, 51 bp, Artificial Sequence
  Synthetic construct
aaaaaaaaaaaataaaaaaaaaaaataaaaaaaaaaaataaaaaaaaaaaa

SEQ ID NO: 2 - DNA, 50 bp, Artificial Sequence
  Synthetic construct
tttttttttttttttttttttttttttttttttttttttttttttttttt

SEQ ID NO: 3 - DNA, 51 bp, Artificial Sequence
  Synthetic construct
ggggggggggggtggggggggggggtggggggggggggtgggggggggggg

SEQ ID NO: 4 - DNA, 48 bp, Artificial Sequence
  Synthetic construct
cccccccccccctccccccccccctccccccccccctccccccccccc

SEQ ID NO: 5 - DNA, 49 bp, Artificial Sequence
  Synthetic construct
ttttttttttttattttttttttttattttttttttatttttttttttt

SEQ ID NO: 6 - DNA, 48 bp, Artificial Sequence
  Synthetic construct
ccccccccccccacccccccccccacccccccccccaccccccccccc

SEQ ID NO: 7 - DNA, 21 bp, Artificial Sequence
  Synthetic construct
ctaggcgtttgtactccgtca

SEQ ID NO: 8 - DNA, 20 bp, Artificial Sequence
  Synthetic construct
tcctcagaagtttatgcact

(19) United States
(12) Patent Application Publication
(10) Pub. No.: US 2021/0277048 A1
(43) Pub. Date: Sep. 9, 2021

(54) METHOD OF PREPARING NUCLEASE-RESISTANT DNA-INORGANIC HYBRID NANOFLOWERS

(71) Applicants: KOREA ADVANCED INSTITUTE OF SCIENCE AND TECHNOLOGY, Daejeon (KR); GACHON UNIVERSITY OF INDUSTRY-ACADEMIC COOPERATION FOUNDATION, Gyeonggi-do (KR)

(72) Inventors: Hyun Gyu PARK, Daejeon (KR); Moon Il Kim, Gyeonggi-do (KR); Ki Soo Park, Daejeon (KR); Chang Yeol Lee, Daejeon (KR); Phuong Thy Nguyen, Gyeonggi-do (KR)

(21) Appl. No.: 17/321,328

(22) Filed: May 14, 2021

Related U.S. Application Data
(63) Continuation-in-part of application No. 15/924,242, filed on Mar. 18, 2018.

(30) Foreign Application Priority Data
May 2, 2017 (KR) 10-2017-0056226

(51) Int. Cl. C07H 23/00; C07H 1/00

(52) U.S. Cl. CPC C07H 23/00; B82Y 40/00; C07H 1/00

(57) ABSTRACT

The present invention relates to a method of preparing nucleic acid-inorganic hybrid nanoflowers, which comprises allowing a nucleic acid to react with a solution of a metal ion-containing compound at room temperature, thereby forming a complex between the metal ion and the nitrogen atom of an amide bond or amine group present in the nucleic acid. According to the present invention, organic-inorganic hybrid nanoflower structures may be synthesized using nucleic acid in a simple manner under an environmentally friendly condition without any toxic chemical substance. The produced organic-inorganic hybrid nanoflower structures show a high DNA encapsulation yield, have resistance against nuclease, and show significantly increased peroxidase activity. Thus, these nanoflower structures may be widely used as a gene therapy carrier and in biosensing technology.

### CROSS-REFERENCE TO RELATED APPLICATIONS

This is a continuation-in-part under 35 USC § 120 of U.S. patent application Ser. No. 15/924,242 filed Mar. 18, 2018 for “METHOD OF PREPARING NUCLEASE-RESISTANT DNA-INORGANIC HYBRID NANO FLOWERS”, which in turn claims priority under 35 USC § 119 of Korean Patent Application 10-2017-0056226 filed May 2, 2017. The disclosures of U.S. patent application Ser. No. 15/924,242 and Korean Patent Application 10-2017-0056226 are hereby incorporated herein by reference, in their respective entireties, for all purposes.

### TECHNICAL FIELD

The present invention relates to a method of preparing nuclease-resistant DNA-inorganic hybrid nanoflowers, and more particularly to a method of preparing nucleic acid-inorganic hybrid nanoflowers, which comprises allowing a nucleic acid to react with a solution of a metal ion-containing compound at room temperature, thereby forming a self-assembled complex between the metal ion and the nitrogen atom of an amide bond or amine group present in the nucleic acid, which resembles flower in nanometer scale.

### BACKGROUND ART

Flower-shaped nanomaterials called nanoflowers have attracted attention in various fields, including catalysis, electronics and analytical chemistry, due to their property of having a rough surface and a large surface-to-volume ratio (A. Mohanty et al., Angew. Chem. Int. Ed. 2010, 5, 4962; J. Xie et al., ACS Nano 2008, 23, 2473; Z. Lin, Y et al., RSC Adv., 2014, 4, 13888). Recently, the Zare research group succeeded in synthesizing organic/inorganic hybrid nanoflowers using various enzymes and proteins with copper sulfate at room temperature, and found that enzymes loaded on the hybrid nanoflowers have higher activity, stability and durability than general enzymes dissolved in aqueous solutions (J. Ge et al., Nanotechnol., 2012, 7, 428). This increased enzymatic activity may be applied to systems that analyze various materials in a highly sensitive and stable manner. Until now, biosensor systems for the detection of phenol, hydrogen peroxide and glucose have been developed (L. Zhu et al., Chem. Asian. J., 2013, 8, 2358; Z. Lin et al., ACS. Appl. Mater. Inter., 2014, 6, 10775; J. Sun et al., Nanoscale, 2014, 6, 225).

A protein that forms the nanoflowers contains many nitrogen atoms in the amide bonds and amine groups, and a possible synthetic mechanism was proposed according to which such moieties form complexes with copper ions via coordination interaction, so that the synthesis of primary copper-protein nanoparticles will be induced, and consequently, nanoflower structures will be formed by time-dependent precipitation (J. Ge, et al., Nat. Nanotechnol., 2012, 7, 428). For example, it was found that organic-inorganic hybrid nanoflowers can be synthesized using various proteins such as bovine serum albumin, a-lactalbumin, laccase, carbonic anhydrase, and lipase (B. S. Batule et al., J. Nanomedicine, 2015, 10, 137). The above-described technology is meaning significant in that it is a new technology of synthesizing nanoflower structures using proteins. However, its expansion to other organic biological molecules has not been reported.

Accordingly, the present inventors have found that nucleic acid incubated with a metal ion-containing compound can induce a hybrid nanoflower, which consists of both nucleic acid and metal compound as organic and inorganic compound, respectively. The incubation is performed at room temperature under an environmentally friendly condition in a very simple manner, and the nucleic acid-inorganic hybrid nanoflowers and the nucleic acid-nanoparticles-inorganic hybrid nanoflowers comprising nanoparticles such as magnetic nanoparticles, thus produced have low cytotoxicity, show significantly increased loading capacities compared to those produced by a conventional DNA loading technology, and have high resistance against nuclease, thereby completing the present invention.

### SUMMARY OF INVENTION

It is an object of the present invention to provide a method of preparing DNA-inorganic hybrid nanoflowers, comprising synthesizing nucleic acid-inorganic hybrid nanoflowers in a very simple manner at room temperature under an environmentally friendly condition without addition of any toxic reducing agents or the like.

Another object of the present invention is to provide nucleic acid-inorganic hybrid nanoflowers that have low cytotoxicity, show high loading capacities, and have high resistance against nuclease.

In addition, another object of the present invention is to provide a nucleic acid-nanoparticles-inorganic hybrid nanoflower that easily captures nanoparticles including magnetic nanoparticles.

The above objects of the present invention can be achieved by the present invention as specified below.

To achieve the above objects, the present invention provides a method of preparing nucleic acid-inorganic hybrid nanoflowers, comprising forming a complex between a metal ion and a nitrogen atom of an amide bond or amine group in the nucleic acid, by reacting the nucleic acid with a solution of the metal ion-containing compound at room temperature.

The present invention also provides nucleic acid-inorganic hybrid nanoflowers having resistance against nuclease, which are produced by the above-described method.

The present invention also provides a carrier for gene therapy, which comprises the above-described nucleic acid-inorganic hybrid nanoflowers.

The present invention also provides a biosensor comprising the above-described nucleic acid-inorganic hybrid nanoflowers.

In addition, the present invention provides a method of preparing a nucleic acid-magnetic nanoparticles-inorganic hybrid nanoflower comprising: (a) reacting nucleic acid and amine-coated magnetic nanoparticles at room temperature to obtain a nucleic acid-magnetic nanoparticle complex bound by electrostatic attraction; and (b) obtaining a nucleic acid-magnetic nanoparticles-inorganic hybrid nanoflower by reacting a solution in which a metal ion-containing compound is dissolved and the nucleic acid-magnetic nanoparticle complex at room temperature to induce a covalent coordination bond between amide bond present in the nucleic acid or nitrogen atom in amine group and nitrogen atom present in amine group and a metal ion on surface of the magnetic nanoparticles.

The present invention also provides nucleic acid-magnetic nanoparticles-inorganic hybrid nanoflower having resistance against nuclease, which are produced by the above-described method.

The present invention also provides a carrier for gene therapy, which comprises the above-described nucleic acid-magnetic nanoparticles-inorganic hybrid nanoflower.

The present invention also provides a biosensor comprising the above-described nucleic acid-magnetic nanoparticles-inorganic hybrid nanoflower.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a process of synthesizing organic-inorganic hybrid nanoflowers using DNA according to an example of the present invention (FIG. 1a), and depicts SEM images showing time-dependent formation of nanoflower structures (FIG. 1b).

FIG. 2 depicts SEM images showing the effect of the DNA concentration on the formation of nanoflower structures according to an example of the present invention.

FIG. 3 depicts SEM images showing the effect of the DNA nucleotide sequence and length on the formation of nanoflower structures according to an example of the present invention.

FIG. 4 depicts electrophoresis images showing the results of analyzing whether DNA loaded on nanoflower structures is degraded by DNase I (FIG. 4A) and exonuclease III (FIG. 4B), which are nucleases, according to an example of the present invention.

FIG. 5 is a graph showing cytotoxicity test results for DNA-inorganic hybrid nanoflower structures synthesized according to an example of the present invention.

FIG. 6 is a graph showing the results of analyzing the peroxidase activity of DNA-inorganic hybrid nanoflower structures synthesized according to an example of the present invention.

FIG. 7 shows a SEM photograph of a DNA-magnetic nanoparticles (MNP)-NF nanoflower structure synthesized according to an example of the present invention. (a) DNA-Cu nanoflower; (b) MNP-Cu nanoflower; and (c) DNA-MNP-Cu nanoflower.

FIG. 8 shows a photograph (b) of magnetic separation of DNA-MNP-Cu nanoflower (a) using a magnet.

FIG. 9 is a graph showing the peroxidase-like activity of the DNA-MNP-Cu nanoflower structure synthesized according to an example of the present invention. (1) DNA-MNP-Cu nanoflower; (2) MNP-Cu nanoflower; (3) DNA-Cu nanoflower; and (4) control.

### BEST MODE FOR CARRYING OUT THE INVENTION

Unless defined otherwise, all the technical and scientific terms used herein have the same meaning as those generally understood by one of ordinary skill in the art to which the invention pertains. Generally, the nomenclature used herein and the experiment methods, which will be described below, are those well-known and commonly employed in the art.

In the present invention, it was found that when a nucleic acid was allowed to react with a solution of a metal ion-containing compound at room temperature, thereby forming a complex between the metal ion and the nitrogen atom of an amide bond or amine group present in the nucleic acid, nucleic acid-inorganic hybrid nanoflowers could be obtained which had resistance against nuclease and in which the DNA loaded on the nanostructures stably maintained its structure even after 24 hours of a sufficient enzymatic reaction.

Therefore, in one aspect, the present invention is directed to a method of preparing nucleic acid-inorganic hybrid nanoflowers, comprising forming a complex between a metal ion and a nitrogen atom of an amide bond or amine group in the nucleic acid, by reacting the nucleic acid with a solution of the metal ion-containing compound at room temperature.

In addition, in another aspect, the present invention is directed to nucleic acid-inorganic hybrid nanoflowers having resistance against nuclease, which are produced by the above-described method.

Based on the principle of synthesis of protein-based nanoflower structures, the present inventors have paid attention to the fact that nucleic acid which is another biopolymer substance also contains many amide bonds and amine groups, and the present inventors expected that the nucleic acid would induce flower-shaped nanostructures, similar to the protein. Based on this expectation, the present inventors carried out experiments, and as a result, have found for the first time that it is possible to synthesize organic-inorganic hybrid nanoflower structures using nucleic acid at room temperature under an environmentally-friendly condition in a very simple manner (K. S. Park, B. S. Batule, K. S. Kang, T. J. Park, M. I. Kim and H. G. Park, J. Mater. Chem. B, 2017, 5, 2231).

The present invention has the following advantages over a conventional method (D. Nykypanchuk et al., Nature, 2008, 451, 553) which uses DNA merely as a linker and only as a template for nanomaterial synthesis: 1) it is possible to synthesize nanoflower structures under an environmentally-friendly condition without addition of any toxic reducing agents; 2) the synthesized DNA-based nanoflower structures have low cytotoxicity; 3) DNA loaded on the nanoflower structures shows significantly increased loading capacities of 95% or more compared to those produced by a conventional DNA loading technology (K. E. Shopsowitz et al., Small, 2014, 10, 1623); and 4) DNA loaded on the nanoflower structures has high resistance against nuclease.

A method of preparing nucleic acid-inorganic hybrid nanoflowers according to the present invention and the nucleic acid-inorganic hybrid nanoflowers produced by the method will be described in detail hereinafter.

In the present invention, the nucleic acid may be DNA or RNA. The metal may be at least one selected from the group consisting of copper (Cu), zinc (Zn), calcium (Ca), and manganese (Mn), and preferably copper is used as the metal, but is not limited thereto.

In addition, in the present invention, the metal ion-containing compound may be at least one selected from the group consisting of copper sulfate (CuSO₄), zinc acetate (Zn(CH₃COO)₂), calcium chloride (CaCl₂)), and manganese sulfate (MnSO₄), and preferably copper sulfate is used as the metal ion-containing compound, but is not limited thereto.

In the method of preparing nucleic acid-inorganic hybrid nanoflowers according to the present invention, the reaction may be performed at room temperature for 60 to 80 hours, and the concentration of the nucleic acid may be 10 μM to 100 μM, preferably 10 μM to 1 μM, depending on the length of the nucleotide sequence thereof. The size of the nucleic acid-inorganic hybrid nanoflowers may be determined depending on the concentration of the nucleic acid.

Further, in the present invention, it was found that the nucleic acid-inorganic hybrid nanoflowers can be utilized in biosensing technology for high-sensitivity detection of target biomaterials as well as can be utilized as a carrier for gene therapy as having no cytotoxicity.

Therefore, in still another aspect, the present invention is directed to a carrier for gene therapy and a biosnesor, which comprises the above-described nucleic acid-inorganic hybrid nanoflowers.

The DNA-inorganic hybrid nanoflower structures produced according to the present invention have no cytotoxicity (100% cell viability), and may be utilized as a carrier for gene therapy in the future. In addition, the DNA-inorganic hybrid nanoflower structures show high peroxidase activity due to their specific large surface area. Furthermore, the DNA-inorganic hybrid nanoflower structures have higher peroxidase activity than that of conventional protein-based nanoflower structures. Thus, the DNA-inorganic hybrid nanoflower structures synthesized according to the present invention may be widely utilized in biosensing technology for high-sensitivity detection of target biomaterials in the future.

In addition, the present invention relates to the development of DNA-MNP-Cu nanoflower in which magnetic nanoparticles easily are trapped, by coating an amine group on the magnetic nanoparticles, and binding the amine-coated magnetic nanoparticles and DNA through an electrostatic attraction, and a reaction with the Cu salt. The DNA-MNP-Cu nanoflower can be easily recovered by external magnetic force due to the characteristics of magnetic nanoparticles, and show more enhanced peroxidase activity due to the peroxidase-like activity of magnetic nanoparticles. In particular, amine-coated nanoparticles other than magnetic nanoparticles can be easily trapped inside DNA-Cu nanoflowers in a similar manner.

Therefore, in another aspect, the present invention is directed to a method of preparing a nucleic acid-magnetic nanoparticles-inorganic hybrid nanoflower comprising: (a) reacting nucleic acid and amine-coated magnetic nanoparticles at room temperature to obtain a nucleic acid-magnetic nanoparticle complex bound by electrostatic attraction; and (b) obtaining a nucleic acid-magnetic nanoparticles-inorganic hybrid nanoflower by reacting a solution in which a metal ion-containing compound is dissolved and the nucleic acid-magnetic nanoparticle complex at room temperature to induce a covalent coordination bond between amide bond present in the nucleic acid or nitrogen atom in amine group and nitrogen atom present in amine group and a metal ion on surface of the magnetic nanoparticles.

Also, in another aspect, the present invention is directed to nucleic acid-magnetic nanoparticles-inorganic hybrid nanoflower having resistance against nuclease, which are produced by the above-described method. In addition, in another aspect, the present invention is directed to a carrier for gene therapy or a biosensor, which comprises the above-described nucleic acid-magnetic nanoparticles-inorganic hybrid nanoflower.

In the present invention, the nucleic acid-magnetic nanoparticle-inorganic hybrid nanoflower can be used as a carrier for gene therapy, and in some cases, can be used as a biosensor.

In the present invention, the kind of therapeutic gene that can be bound to the carrier for gene therapy is not particularly limited, and includes any type of gene capable of exerting a desired therapeutic effect by delivering to a desired target according to the object of the present invention, for example, gDNA, cDNA, pDNA, mRNA, tRNA, rRNA, siRNA, shRNA, miRNA, PNA, and the like, but it is not limited thereto.

In the present invention, the carrier for gene therapy may be administered by an appropriate method, together with a pharmaceutically acceptable carrier. The carrier for gene therapy of the present invention can be variously formulated in the form of an oral formulation or a sterile injectable solution according to a conventional method, and can be prepared as solid nanoparticles and microsphere powders.

In the example as the carrier for gene therapy of the present invention, the gene to be treated can be delivered by binding to a nucleic acid-magnetic nanoparticles-inorganic hybrid nanoflower.

In the case of existing agent for gene therapy, there is a problem in that the therapeutic agent is degraded by the nuclease present in the living body so that the original efficacy cannot be displayed. However DNA nanoflower or DNA-nanoparticles-nanoflower according to the present invention have high resistance to nucleases, and thus can be effectively used as a carrier for gene therapy.

In the carrier for gene therapy of the present invention, the gene to be treated and the nucleic acid inside the nanoflower may be the same, or it may be inactivated by complementary binding to the gene to be treated.

In addition, the nucleic acid-magnetic nanoparticles-inorganic hybrid nanoflower of the present invention can be applied to a system that analyzes a variety of substances very sensitively and stably, and as a biosensor for detecting target biological substances such as phenol, hydrogen peroxide, and glucose with high sensitivity.

The biosensor of the present invention may include a biomolecule recognition material bound to the nucleic acid-magnetic nanoparticles-inorganic hybrid nanoflower.

In this case, the magnetic nanoparticles serve to connect the hybrid nanoflower and the biomolecule recognition material.

The method of binding the biomolecule recognition material to the hybrid nanoflower is not particularly limited, and for example, a method of contacting the hybrid nanoflower and the biomolecule recognition material through a suitable solvent such as PBS may be used.

The biomolecule is a molecule constituting an organism and refers to a molecule necessary for the structure, function, and information transmission of an organism.

The biomolecule recognition substance refers to a biomolecule or other chemical substance capable of specifically binding to a biomolecule to be detected, and examples thereof include at least one substance capable of specifically binding to antigens, antibodies, RNA, DNA, hapten, avidin, streptavidin, neutravidin, protein A, protein G, lectin, selectin, radioisotope marker, aptamer, and tumor marker, but it is not limited thereto.

When the tumor marker is an antigen, a substance capable of specifically binding to the antigen such as a receptor or antibody capable of specifically binding to the antigen, may be introduced into the biomolecule recognition material. Examples of receptors or antibodies capable of specifically binding to the antigen include EGF (epidermal growth factor) and anti-EGFR (ex. cetuximab), synaptotagmin C2 and phosphatidylserine, annexin V and phosphatidylserine, integrin and a receptor thereof, VEGF (Vascular Endothelial Growth Factor) and a receptor thereof, angiopoietin and Tie2 receptor, somatostatin and a receptor thereof or vasointestinal peptide, carcinoembryonic antigen (colorectal cancer marker antigen) and Herceptin (Genentech, USA), HER2/neu antigen (breast cancer marker antigen) and Herceptin, prostate-specific membrane antigen (prostate cancer marker antigen) and rituxan (IDCE/Genentech, USA) and a receptor thereof, but they are not limited thereto.

A representative example in which the tumor marker is a receptor is the folic acid receptor expressed in ovarian cancer cells. A substance capable of specifically binding to the receptor (folic acid in the case of a folic acid receptor) may be introduced into the biosensor according to the present invention, and an example of which include antigens or antibodies capable of specifically binding to the receptor.

As described above, in the present invention the antibody is a particularly preferred tissue-specific binding material and the antibody includes a polyclonal antibody, a monoclonal antibody, and an antibody fragment. Antibodies have the property of selectively and stably binding only to specific targets, and —NH₂ of lysine, —SH of cysteine, —COOH of aspartic acid and glutamic acid in the Fc region of the antibody can be usefully used to bind the antibody to the biosensor of the present invention.

These above antibodies are commercially available or can be prepared according to methods known in the art.

Meanwhile, the nucleic acid includes RNA and DNA encoding the aforementioned antigens, receptors, or portions thereof. Since a nucleic acid has a characteristic of forming a base pair between complementary sequences, a nucleic acid having a specific nucleotide sequence can be detected using a nucleic acid having a nucleotide sequence complementary to the nucleotide sequence. A nucleic acid having a base sequence complementary to the nucleic acid encoding the antigen or receptor can be used in the biosensor according to the present invention.

The nucleic acid has a functional group such as —NH₂, —SH or —COOH at the 5′- and 3′-terminals, and the functional group can be usefully used to bind the nucleic acid to the hybrid nanoflower of the present invention.

Such nucleic acids can be synthesized using standard methods known in the art, for example an automatic DNA synthesizer.

In an example applied as the biosensor of the present invention, a biomolecule recognition material may be sensed by binding to a nucleic acid-magnetic nanoparticles-inorganic hybrid nanoflower.

Since the DNA nanoflower or DNA-nanoparticles-nanoflower of the present invention has excellent peroxidase-mimicking activity, it can be used as a signal material for immunodiagnosis along with the diagnosis of H₂O₂ and various small molecule substances based on peroxidase activity, and in particular, DNA-nanoparticle-nanoflower can be used in a wide range of diagnostic fields by simultaneously utilizing the properties of nanoparticles (magnetism, fluorescence, catalyst, etc.).

The method of preparing a nucleic acid-magnetic nanoparticles-inorganic hybrid nanoflower according to the present invention can form a complex by reacting nucleic acid and amine-coated magnetic nanoparticles at room temperature to binding by electrostatic attraction and reacting a solution in which a metal ion-containing compound is dissolved and the nucleic acid-magnetic nanoparticle complex at room temperature for 3 days to induce a covalent coordination bond between amide bond present in the nucleic acid or nitrogen atom in amine group and nitrogen atom present in amine group and a metal ion on surface of the magnetic nanoparticles.

The DNA-MNP-Cu nanoflower according to an embodiment of the present invention can be easily recovered by external magnetic force due to the characteristics of magnetic nanoparticles, and exhibits more improved peroxidase activity due to the peroxidase-like activity of the magnetic nanoparticles. In particular, amine-coated nanoparticles other than magnetic nanoparticles can be easily trapped inside DNA-Cu nanoflower in a similar manner.

In the present invention, the magnetic nanoparticles may be at least one selected from the group consisting of Fe₃O₄ and Fe₂O₃, and the size of the magnetic nanoparticles may be 10 nm to 20 nm.

In the present invention, in steps (a) and (b), the reaction may be performed at room temperature for 60 to 80 hours, preferably at room temperature for about 3 days.

In the present invention, the nucleic acid and the metal ion-containing compound are the same as those described in the method for preparing the nucleic acid-inorganic hybrid nanoflower and the nucleic acid-inorganic hybrid nanoflower prepared by the method.

### EXAMPLES

Hereinafter, the present invention will be described in further detail with reference to examples. It will be obvious to a person having ordinary skill in the art that these examples are for illustrative purposes only and are not to be construed to limit the scope of the present invention.

### Example 1: Production of Nuclease-Resistant DNA-Inorganic Hybrid Nanoflowers

DNAs having various nucleotide sequences and lengths were allowed to react with copper sulfate at room temperature for 3 days, thereby producing DNA-inorganic hybrid nanoflowers.

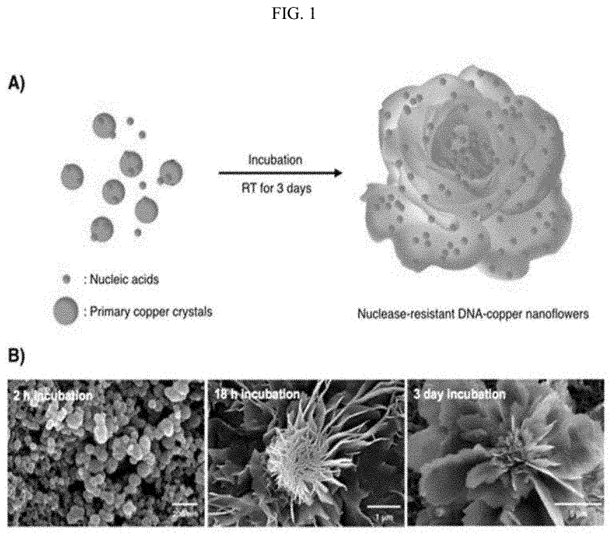

FIG. 1(a) shows a process of synthesizing organic-inorganic hybrid nanoflower structures using DNA. When DNAs having various nucleotide sequences and lengths are allowed to react with copper sulfate at room temperature for 3 days, nanoflower structures having large surface areas are obtained. In the principle of synthesis, the nitrogen atoms in amide bonds or amide groups present in the nucleic acids form a complex with copper ions, similar to proteins, whereby flower-shaped structures are synthesized. FIG. 1(b) depicts SEM (scanning electron microscope) images showing the time-dependent formation of nanoflower structures. As can be seen in FIG. 1(b), in the initial reaction stage (2 hours), small flower bud shapes were formed, and with the passage of time (18 hours), flower shapes were formed, and finally after 3 days, complete flower-shaped structures having a large surface-to-volume ratio were formed. This process of forming DNA-based nanoflower structures is similar to a process of forming nanoflowers using protein (J. Ge et al., Nanotechnol., 2012, 7, 428).

### Example 2: Production of DNA-Inorganic Hybrid Nanoflowers Using Various Concentrations of DNA

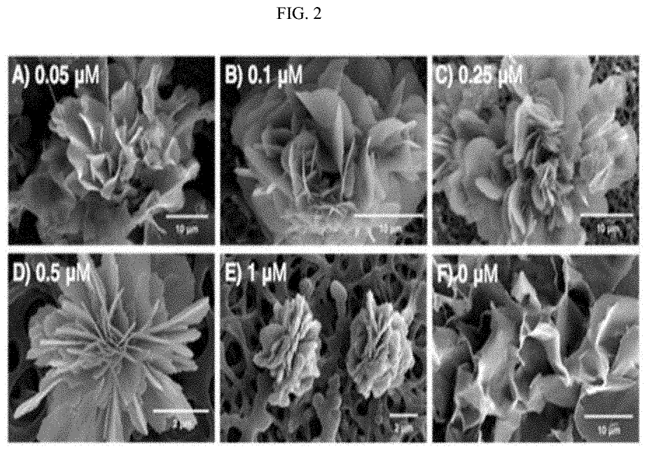

The effect of the DNA concentration on the production of DNA-inorganic hybrid nanoflowers was examined. FIG. 2 depicts SEM images showing the results of an experiment performed to examine the effect of the DNA concentration (A: 0.05 μM, B: 0.1 μM, C: 0.25 μM, D: 0.5 μM, E: 1 μM, F: 0 μM) on the formation of nanoflower structures. As can be seen in FIG. 2, when the DNA concentration was low, relatively large nanoflower structures having an average size of about 30 μm were formed (FIGS. 2A, 2B and 2C). However, when the DNA concentration was high, relatively small nanoflower structures having an average size of about 5 μm were formed (FIGS. 2D and 2E). In addition, it could be seen that such nanoflower structures were formed only in the presence of the DNA (FIG. 2F).

### Example 3: Production DNA-Inorganic Hybrid Nanoflowers Using Various DNA Nucleotide Sequences and Lengths

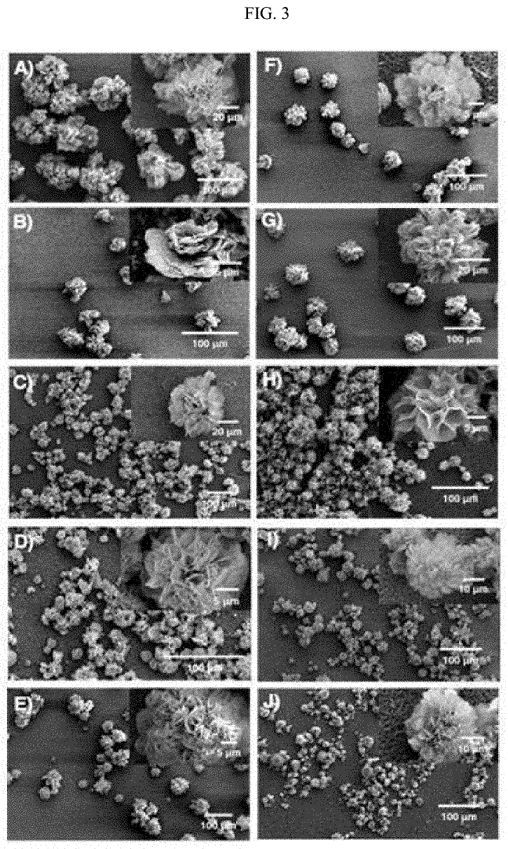

The effect of the DNA nucleotide sequence and length on the production of DNA-inorganic hybrid nanoflowers was examined. FIG. 3 depicts SEM images showing the results of an experiment performed to the effect of the DNA nucleotide sequence and length on the formation of nanoflower structures. Information including the DNA nucleotide sequences used in the experiment is shown in Table 1 below. As can be seen in FIG. 3, all the DNAs (A: dNTPs, B: Adenine-rich single-stranded (ss) DNA, C: Thymine-rich ssDNA, D: Guanine-rich ssDNA, E: Cytosine-rich ssDNA, F: 51-bp Adenine-Thymine double-stranded (ds) DNA, G: 51-bp Guanine-cytosine dsDNA, H: 200-bp PCR amplicon, I: 5420-bp plasmid DNA, J: 4857-kbp genomic DNA) used in the experiment formed flower structures having an average size of 20 to 50 μm, and the DNA encapsulation yield of the produced flower structures was 95% or higher (Table 2). Here, the DNA encapsulation yield is defined as the ratio of the amount of DNA loaded on nanoflower structures to the amount of DNA introduced in the initial stage. In addition, it could be seen that the weight percentage of the loaded DNA in entire nanoflower structures was 7 to 13%, which was similar to that in conventional protein-based flower structures (Table 2, Lin, Y. Xiao et al., ACS. Appl. Mater. Inter., 2014, 6, 10775).

TABLE 1

| DNA samples | Sequences or information |
|---|---|
| A)dNTPs | dATP, dTTP, dGTP and dCTP |
| B)Adenine-rich | 5′-AAA AAA AAA AAA TAAA AAA |
| ssDNA | AAA AAA TAAA AAAAAAAAA TAAA |
| | AAA AAA AAA-3′ |
| | (SEQ ID NO: 1) |
| C)Thymine-rich | 5′-TTT TTT TTT TTT T TTT TTT |
| ssDNA | TTT TTT T TTT TTT TT TTT T |
| | TTT TTT TTT TTT-3′ |
| | (SEQ ID NO: 2) |
| D)Guanine-rich | 5′-GGG GGG GGG GGG T GGG GGG |
| ssDNA | GGG GGG T GGG GGG GGG GGG T |
| | GGG GGG GGG GGG-3′ |
| | (SEQ ID NO: 3) |
| E)Cytosine-rich | 5′-CCC CCC CCC CCC TCC CCC |
| ssDNA | CCC CCC TCC CCC CCC CCC TCC |
| | CCC CCC CCC-3′ |
| | (SEQ ID NO: 4) |
| F)ssDNA | 5′-TTT TTT TTT TTT A TTT TTT |
| complementary | TTT TTT A TTT TTT T TTT A TTT |
| to B for A-T | TTT TTT TTT-3′ |
| dsDNA | (SEQ ID NO: 5) |
| G)ssDNA | 5′-CCC CCC CCC CCC ACC CCC |
| complementary | CCC CCC ACC CCC CCC CCC ACC |
| to D for G-C | CCC CCC CCC-3′ |
| dsDNA | (SEQ ID NO: 6) |
| H)PCR amplicon | Sample was obtained by |
| (200 bp) | amplifying the |
| | genomic DNA of |
| | Chlamydia trachomatis |
| | using the following |
| | primers. |
| | Forward primer: 5′- |
| | CTA GGC GTT TGT ACT CCG |
| | TCA-3′ |
| | (SEQ ID NO: 7) |
| | Reverse primer: 5′- |
| | TCC TCA GAA GTT TAT GCA CT-3′ |
| | (SEQ ID NO: 8) |
| I)Plasmid DNA | pETDuet-1 |
| (5420 bp) | |
| J)Genomic DNA | Sample was obtained by |
| (4857 bp) | purifying the genomic |
| | DNA of Salmonella |
| | typhimurium. |

TABLE 2

| DNA samples | Encapsulation yield(%) | Weight percentage(%) |
|---|---|---|
| A)dNTPs | 97 | 13 |
| B)Adenine-rich ssDNA | 97 | 10 |
| C)Thymine-rich ssDNA | 99 | 7 |
| D)Guanine-rich ssDNA | 99 | 9 |
| E)Cytosine-rich ssDNA | 99 | 9 |
| F)A-T dsDNA (51 bp) | 98 | 8 |
| G)G-C dsDNA (51 bp) | 98 | 7 |
| H)PCR amplicon (200 bp) | 95 | 10 |
| I)Plasmid DNA (5420 bp) | 99 | 9 |
| J)Genomic DNA (4857 bp) | 97 | 10 |

### Example 4: Examination of Applicability of DNA-Inorganic Hybrid Nanoflowers

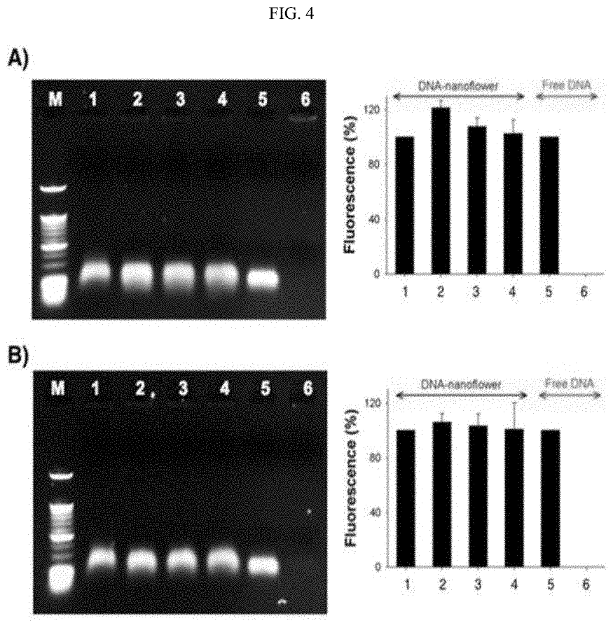

FIG. 4 depicts experimental results indicating that the produced DNA-inorganic hybrid nanoflower structures have resistance against nucleases in an experiment performed to examine the applicability of the DNA-inorganic hybrid nanoflower structures. The experiment was performed to determine whether the DNA loaded on the nanoflower structures would be degraded by typical nucleases, DNase I (FIG. 4A) and exonuclease III (FIG. 4B), and the results of the experiment were confirmed by electrophoresis. As can be seen in FIG. 4, free DNA was completely degraded by the nucleases (lane 5: DNA before reaction; and lane 6: DNA that reacted with DNase I (FIG. 4A) or Exonuclease III (FIG. 4B) for 30 minutes). However, it could be seen that the DNA loaded on the nanoflower structures had resistance against the two kinds of nucleases and stably maintained its structure even after 24 hours of a sufficient enzymatic reaction (lane 1: DNA-inorganic hybrid nanoflower structures before reaction, lane 2: DNA-inorganic hybrid nanoflower structures reacted with DNAse I (A) or Exonuclease III (B) enzyme for 30 mins, lane 3: DNA-inorganic hybrid nanoflower structures reacted with DNAse I (A) or Exonuclease III (B) enzyme for 6 hrs, lane 4: DNA-inorganic hybrid nanoflower structures reacted with DNAse I (A) or Exonuclease III (B) enzyme for 24 hrs).

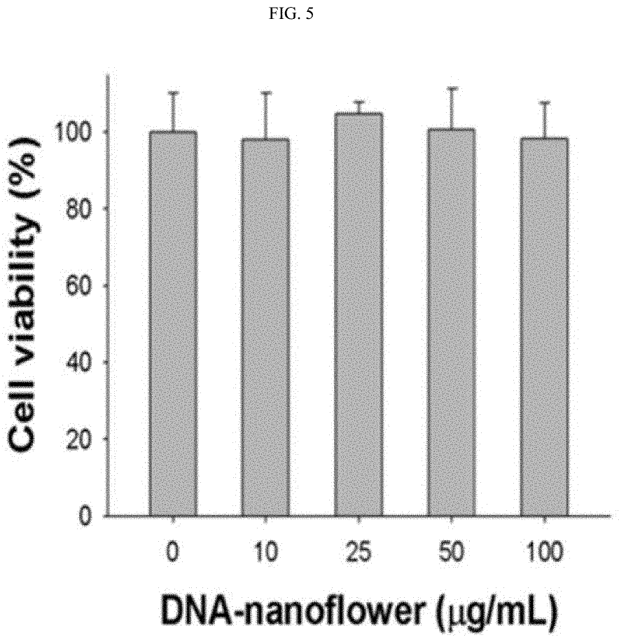

In addition, various concentrations of the DNA-inorganic hybrid nanoflower structures were introduced into cells which were then incubated for 24 hours, after which the effect of the toxicity of the nanoflower structures on the cells was analyzed. The results of the analysis are shown in FIG. 5. As shown in FIG. 5, it could be seen that the DNA-inorganic hybrid nanoflower structures had no cytotoxicity (100% cell viability). Based on such excellent characteristics, the DNA-inorganic hybrid nanoflower structures are expected to be utilized as a carrier for gene therapy in the future.

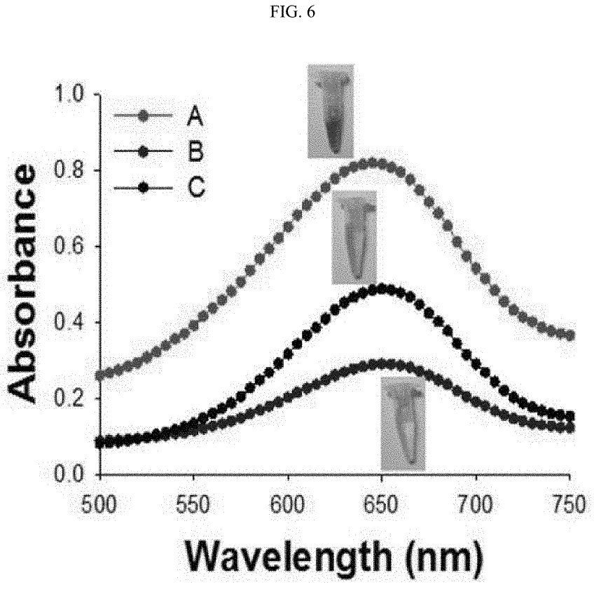

Furthermore, the peroxidase activity of the synthesized DNA-inorganic hybrid nanoflower structures was analyzed, and the results of the analysis are shown in FIG. 6. As can be seen in FIG. 6, when DNA was absent, a precipitate formed from the copper sulfate salt showed a very low peroxidase activity (FIG. 6B). However, it could be seen that the nanoflower structures synthesized by the reaction between the DNA and the copper sulfate salt showed high peroxidase activity due to their large surface area (FIG. 6A). Furthermore, it was found that the DNA-inorganic hybrid nanoflower structures had higher peroxidase activity than that of conventional protein-based nanoflower structures. Thus, the DNA-inorganic hybrid nanoflower structures synthesized according to the present invention is expected to be widely utilized in biosensing technology for high-sensitivity detection of target biomaterials in the future.

### Example 5: Preparation of DNA-MNP-Cu Nanoflower

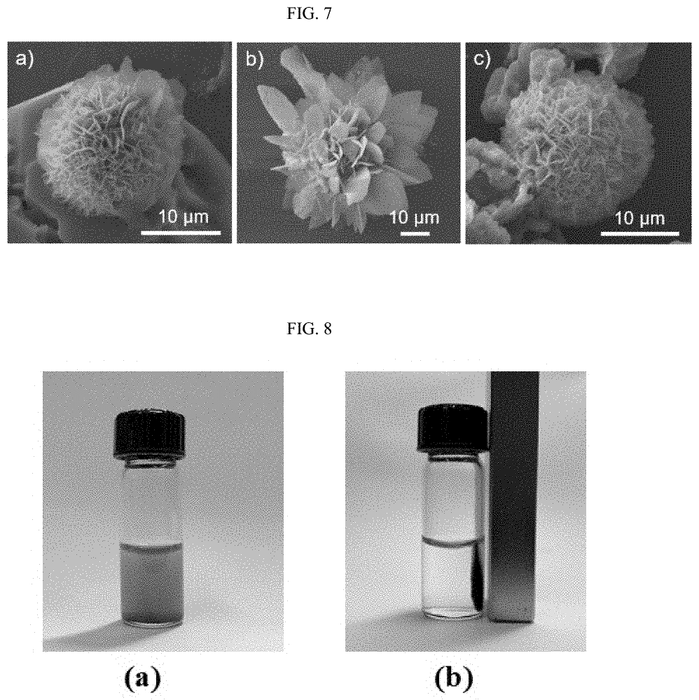

FIG. 7 shows an SEM image of a DNA-MNP-Cu nanoflower and an SEM image of a conventional DNA-Cu nanoflower and MNP-Cu nanoflower according to an example of the present invention.

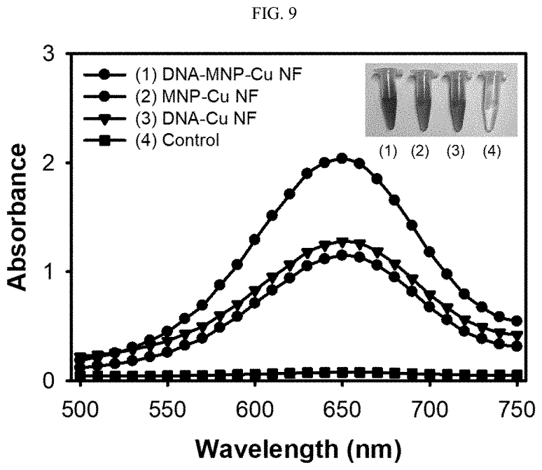

FIG. 8 shows the property that DNA-MNP-Cu nanoflower according to an example of the present invention is recovered by an external magnetic force.

FIG. 9 shows that DNA-MNP-Cu nanoflower according to an example of the present invention exhibit improved peroxidase activity compared to the conventional DNA-Cu nanoflower or MNP-Cu nanoflower.

### Example 5-1: Preparation of Amine-Coated Magnetic Nanoparticles

(I) Synthesis of Magnetic Nanoparticles

For the preparation of DNA-MNP-Cu nanoflowers, magnetic nanoparticles (particle size 10-20 nm) were first synthesized through hydrothermal treatment. The synthesis of magnetic nanoparticles was prepared through co-precipitation of FeCl₃ and FeCl₂ (Mehta et al. Biotechnology Techniques, 11(7), 493-496, 1997). The magnetic nanoparticles uniformly precipitated were sufficiently dried in a vacuum oven, and the size and image thereof were confirmed by TEM.

(II) Synthesis of Amine-Coated Magnetic Nanoparticles

0.5 g of the magnetic nanoparticles prepared in (I) were added to 100 mL of a solution in which ethanol and toluene were mixed at 1:1 (v/v) and dispersed, and then APTES (100 μL of 3-aminopropyl triethoxysilane) solution was added and coated to obtain amine-coated magnetic nanoparticles.

### Example 5-2: Preparation of Nanoflower Containing Magnetic Nanoparticles and DNA (DNA-MNP-Cu NF)

(I) Synthesis of Magnetic Nanoparticles-Nanoflower (MNP-Cu NF)

4 mL of PBS buffer, MNP-APTES (prepared in Example 5-1 (II)) with 0.4 mg/mL concentration, pH 7.4 and 20 μL of 120 mM CuSO₄ were mixed and followed by incubation at room temperature for 3 days. The synthesized nanoflower was washed three times with DI water, and then MNP-Cu NF was obtained.

(II) Synthesis of DNA-Magnetic Nanoparticles-Cu Nanoflower (DNA-MNP-Cu NF)

4 mL of PBS buffer, MNP-APTES (prepared in Example 5-1 (II)) at a concentration of A-rich ssDNA 0.1 μM and 0.4 mg/mL, pH 7.4 and 20 μL of 120 mM CuSO₄ were mixed and followed by incubation at room temperature for 3 days. The synthesized nanoflower was washed three times with DI water, and then MNP-Cu NF was obtained. It was confirmed from the SEM image that the images of the conventional DNA-Cu nanoflower and MNP-Cu nanoflower are different to each other (FIG. 7). In addition, it was confirmed that the synthesized DNA-MNP-Cu nanoflower can be recovered simply by using a magnet, thereby maintaining magnetic properties (FIG. 8). In addition, it was confirmed that the DNA-MNP-Cu nanoflower showed more improved peroxidase activity (FIG. 9).

### INDUSTRIAL APPLICABILITY

The method of preparing nucleic acid-inorganic hybrid nanoflower and nucleic acid-magnetic nanoparticles-inorganic hybrid nanoflower according to the present invention has an effect in that nucleic acid-inorganic hybrid nanoflowers can be synthesized in a very simple manner using the nucleic acid at room temperature under an environmentally friendly condition without addition of any toxic reducing agents or the like. The nucleic acid-inorganic hybrid nanoflower and nucleic acid-magnetic nanoparticles-inorganic hybrid nanoflower thus produced have low cytotoxicity, show significantly increased loading capacities of 95% or more compared to those produced by a conventional DNA loading technology, and have high resistance against nuclease, so that they can have high utilization value as a carrier for gene therapy and a biosensor for high-sensitivity detection of target biomaterials.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof

What is claimed is:

1. A method of preparing a nucleic acid-magnetic nanoparticles-inorganic hybrid nanoflower comprising:
(a) reacting nucleic acid and amine-coated magnetic nanoparticles at room temperature to obtain a nucleic acid-magnetic nanoparticle complex bound by electrostatic attraction; and
(b) obtaining a nucleic acid-magnetic nanoparticles-inorganic hybrid nanoflower by reacting a solution in which a metal ion-containing compound is dissolved and the nucleic acid-magnetic nanoparticle complex at room temperature to induce a covalent coordination bond between amide bond present in the nucleic acid or nitrogen atom in amine group and nitrogen atom present in amine group and a metal ion on surface of the magnetic nanoparticles.

2. The method of preparing nucleic acid-magnetic nanoparticles-inorganic hybrid nanoflower of claim 1, wherein the nucleic acid is DNA or RNA.

3. The method of preparing nucleic acid-magnetic nanoparticles-inorganic hybrid nanoflower of claim 1, wherein the metal is at least one selected from the group consisting of copper (Cu), zinc (Zn), calcium (Ca) and manganese (Mn).

4. The method of preparing nucleic acid-magnetic nanoparticles-inorganic hybrid nanoflower of claim 1, wherein the magnetic nanoparticle is at least one selected from the group consisting of Fe₃O₄ and Fe₂O₃.

5. The method of preparing nucleic acid-magnetic nanoparticles-inorganic hybrid nanoflower of claim 4, wherein size of the magnetic nanoparticles is 10 nm to 20 nm.

6. The method of preparing nucleic acid-magnetic nanoparticles-inorganic hybrid nanoflower of claim 1, wherein the metal ion-containing compound is at least one selected from the group consisting of copper sulfate (CuSO₄), zinc acetate (Zn(CH₃COO)₂), calcium chloride (CaCl₂) and manganese sulfate (MnSO₄).

7. The method of preparing nucleic acid-magnetic nanoparticles-inorganic hybrid nanoflower of claim 1, wherein a reaction in steps of (a) and (b) is performed at room temperature for 60 to 80 hours.

8. The method of preparing nucleic acid-magnetic nanoparticles-inorganic hybrid nanoflower of claim 1, wherein a concentration of the nucleic acid is 10 μM to 1 μM.

9. A nucleic acid-magnetic nanoparticles-inorganic hybrid nanoflower having resistance against nuclease, which are produced by the method of claim 1.

10. The nucleic acid-magnetic nanoparticles-inorganic hybrid nanoflower of claim 9, wherein a weight percentage of the nucleic acid in total nanoflowers is 7 to 13 wt %.

11. A carrier for gene therapy, which comprises the nucleic acid-magnetic nanoparticles-inorganic hybrid nanoflower of claim 8.
